(19) United States
(12) Patent Application Publication
(10) Pub. No.: US 2025/0000663 A1
(43) Pub. Date: Jan. 2, 2025

(54) GLENOID IMPLANTS

(71) Applicant: HOWMEDICA OSTEONICS CORP., Mahwah, NJ (US)

(72) Inventors: Lance Nathan TERRILL, Cork (IE); Garrett CAMPBELL, Huntington, NY (US)

(73) Assignee: HOWMEDICA OSTEONICS CORP., Mahwah, NJ (US)

(21) Appl. No.: 18/576,399

(22) PCT Filed: Jun. 21, 2022
(86) PCT No.: PCT/US2022/034245

§ 371 Date: Jan. 4, 2024

Related U.S. Application Data
(60) Provisional application No. 63/223,694, filed on Jul. 20, 2021.

(51) Int. Cl. A61F 2/40; A61F 2/30

(52) U.S. Cl. CPC A61F 2/4081; A61F 2002/30772

(57) ABSTRACT

Various embodiments of novel glenoid implant are disclosed.

### CROSS-REFERENCE TO RELATED APPLICATION

This application claims priority to U.S. Provisional Application No. 63/223,694, filed Jul. 20, 2021, the entirety of which is incorporated by reference herein.

### FIELD OF DISCLOSURE

The present disclosure generally relates to glenoid implants for shoulder prosthesis.

### BACKGROUND

A shoulder prosthesis includes a glenoid implant intended to replace the glenoid cavity of the scapula and/or a humeral implant intended to replace the humeral head. The glenoid implant generally includes and articular body intended to articulate with the humeral head, and a fixation means to stabilize the articular body with respect to the scapula.

Conventional revisable glenoid implants utilizing metal baseplate design uses bone screws for baseplate fixation. However, bone screws have limited potential for long-term fixation, because bone is viscoelastic and relaxes over time. The typical use of bone screws is in an array of 4-5 mm diameter screws. This array is not only expensive and requires significant intraoperative time to prepare and guide the screw trajectories, but also the clinical literature shows occasional screw breakage below the screw head.

Thus, improved glenoid implant design that offers enhanced and durable primary fixation to the bone is desired.

### SUMMARY

Provided herein are various embodiments of a glenoid implant that provides a bearing surface for the glenoid that incorporate a large screw design, having a diameter from 5-20 mm, to create enhanced fixation strength with the glenoid than using conventional bone screws.

Also provided is a glenoid implant for implantation in a glenoid, the implant comprising: an articular body comprising an articulation surface and an anchoring surface on opposite side of the articular body; at least one protrusion integrally formed with the articular body and extending from the anchoring surface; and a fixation element provided for each of the at least one protrusion, wherein each fixation element comprising: a proximal end, a distal end, a longitudinally-extending recess extending distally from the proximal end, and a screw-threaded external surface, wherein the at least one protrusion and the corresponding fixation element are configured for snap-fitting engagement when the at least one protrusion is inserted into the longitudinally-extending recess of the fixation element.

Also provided is a glenoid implant comprising: an articular body comprising an articulation surface and an anchoring surface on opposite side of the articular body; two protrusions that are integrally formed with the articular body and extending from the anchoring surface; and one fixation element provided for each of the two protrusions, wherein each fixation element comprising: a proximal end, a distal end, a longitudinally-extending recess extending distally from the proximal end, and a screw-threaded external surface, wherein the two protrusions and the corresponding fixation element are configured for snap-fitting engagement when the two protrusions are inserted into the close-ended recess of the corresponding fixation element.

### BRIEF DESCRIPTION OF THE DRAWINGS

The various embodiments of the inventive hydrogel implant of the present disclosure will be described in more detail in conjunction with the following drawing figures. The structures in the drawing figures are illustrated schematically and are not necessarily intended to show actual dimensions or relative scale.

FIG. 1A is an isometric view of a glenoid implant according an embodiment of the present disclosure.

FIG. 1B is a cross-sectional view of the glenoid implant of FIG. 1A.

FIG. 1C is a side view of a protrusion 120 extending from the anchoring surface of the glenoid implant of FIG. 1A showing the profile of the protrusion.

FIG. 1D is a cross-sectional view of a fixation element 130.

FIG. 2A is an isometric view of a glenoid implant according to another embodiment of the present disclosure.

FIG. 2B is another isometric view of the glenoid implant of FIG. 2A.

FIG. 2C is a sideview of the glenoid implant of FIG. 2A.

FIG. 2D is a cross-sectional view of the glenoid implant of FIG. 2A.

FIG. 2E is a sideview of a protrusion 220 extending from the anchoring surface of the glenoid implant of FIG. 2A showing the profile of the protrusion.

FIG. 2F is a cross-sectional view of the fixation element 230, 230′.

FIG. 2G is a cross-sectional view of another embodiment of the glenoid implant of FIG. 2A.

FIG. 2H is an isometric view of the glenoid implant of FIG. 2A, without the fixation elements 230, 230′.

### DETAILED DESCRIPTION

This description of the exemplary embodiments is intended to be read in connection with the accompanying drawings, which are to be considered part of the entire written description. The drawing figures are not necessarily to scale and certain features may be shown exaggerated in scale or in somewhat schematic form in the interest of clarity and conciseness. In the description, relative terms such as “horizontal,” “vertical,” “up,” “down,” “top” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise. When only a single machine is illustrated, the term “machine” shall also be taken to include any collection of machines that individually or jointly execute a set (or multiple sets) of instructions to perform any one or more of the methodologies discussed herein. The term “operatively connected” is such an attachment, coupling or connection that allows the pertinent structures to operate as intended by virtue of that relationship. In the claims, means-plus-function clauses, if used, are intended to cover the structures described, suggested, or rendered obvious by the written description or drawings for performing the recited function, including not only structural equivalents but also equivalent structures.

Provided herein are various improved glenoid implants that have articulation surface that is configured to engage with an anatomical humeral head or a humeral component of a shoulder replacement implant system. Therefore, references to “a humeral head” as used herein should be construed to include both an anatomical humeral head as well as implant humeral head.

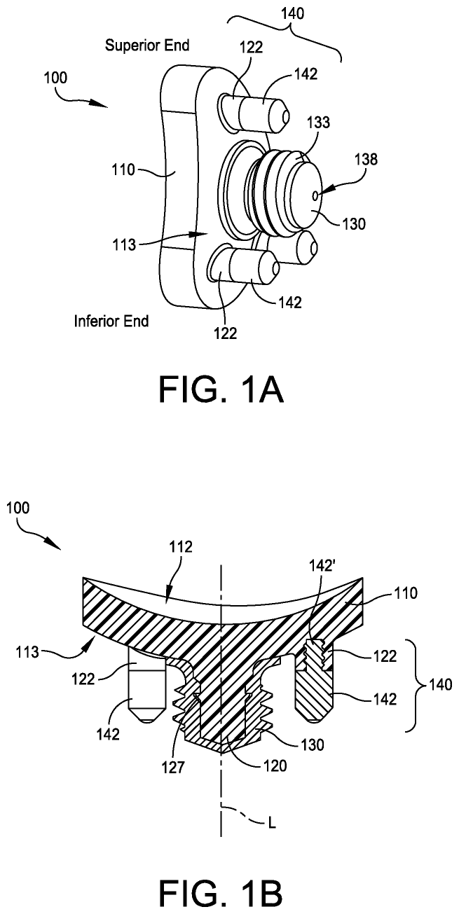

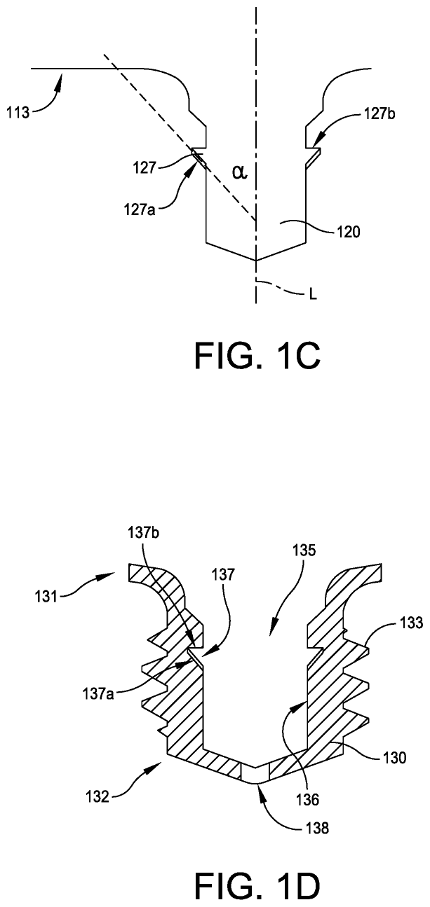

Referring to FIGS. 1A-1D, an improved glenoid implant 100 for implantation in a glenoid according to the present disclosure is provided. The implant 100 comprises an articular body 110, at least one protrusion 120 integrally formed with the articular body, and a fixation element or a hub 130 provided for each of the at least one protrusion. The fixation element 130 is configured to receive a corresponding protrusion 120. The fixation element can be first inlaid into the glenoid, then the articular body is installed in place by inserting the corresponding protrusion 120 into the fixation element 130 and securing it in place.

The articular body 110 comprises an articulation surface 112 and an anchoring surface 113 on the opposite side of the articulation body. The at least one protrusion 120 extends from the anchoring surface 113. Each of the fixation element 130 comprises a proximal end 131 (proximal with respect to the articular body 110), a distal end 132 (distal with respect to the articular body 110), a longitudinally-extending recess 135, for receiving the corresponding protrusion 120, extending distally from the proximal end, and a screw-threaded external surface 133. The longitudinally-extending recess 135 can be open-ended and in some embodiments, it can be close-ended. The screw threads on the external surface 133 can be self-tapping threads.

Each of the fixation element and the corresponding protrusion 120 can be configured to engage each other securely. In some examples, the longitudinally-extending recess 135 and the corresponding protrusion 120 can be configured for a snap-fitting engagement when the protrusion is inserted into the recess 135.

In some embodiments, each of the at least one protrusion 120 can comprise one or more substantially radially extending tabs 127 provided on the exterior surface of the protrusion 120 and the recess 135 in each of the fixation element 130 can comprise a corresponding annular groove 137, where the one or more substantially radially extending tabs 127 and the annular groove 137 can engage each other to form the snap-fitting engagement.

In some embodiments, the snap-fitting engagement can be achieved by the use of a C-clip or an O-ring. For example, instead of the one or more substantially radially extending tabs 127 on the exterior surface of the protrusion 120, a c-clip or an O-ring can be provided over a recess that circumscribes the exterior surface of the protrusion 120 so that when the protrusion 120 is inserted into the recess 135, the c-clip or the O-ring gets captured between the recess on the exterior surface of the protrusion 120 and the annular groove 137 thus forming the snap-fitting engagement.

In the embodiments in which the recess 135 is a close-ended recess, the fixation element 130 can further comprise a guide hole 138 that is used to guide the fixation element 130 over a guide pin during the implant surgical procedure which is described below.

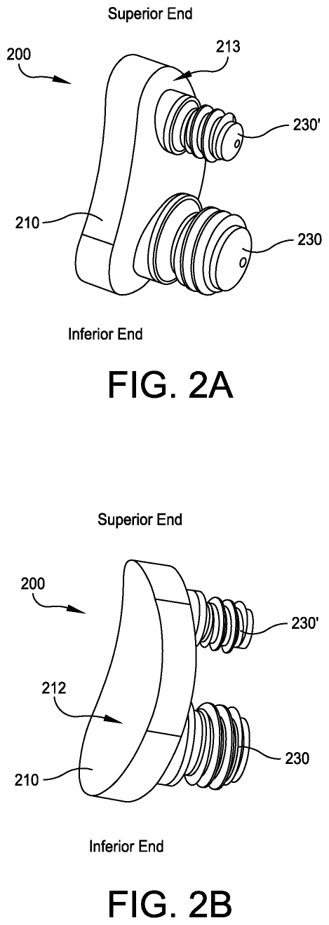

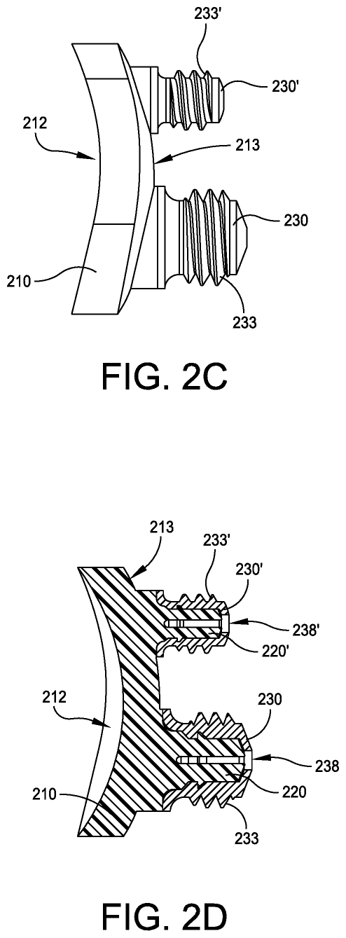

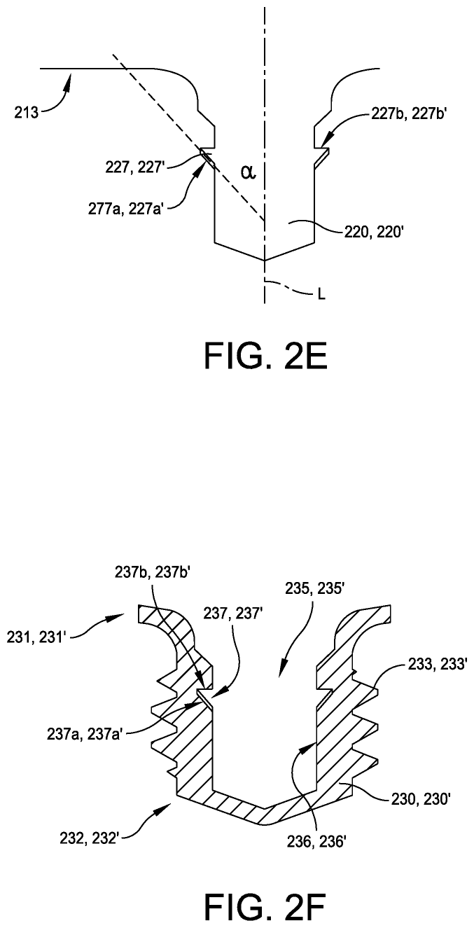

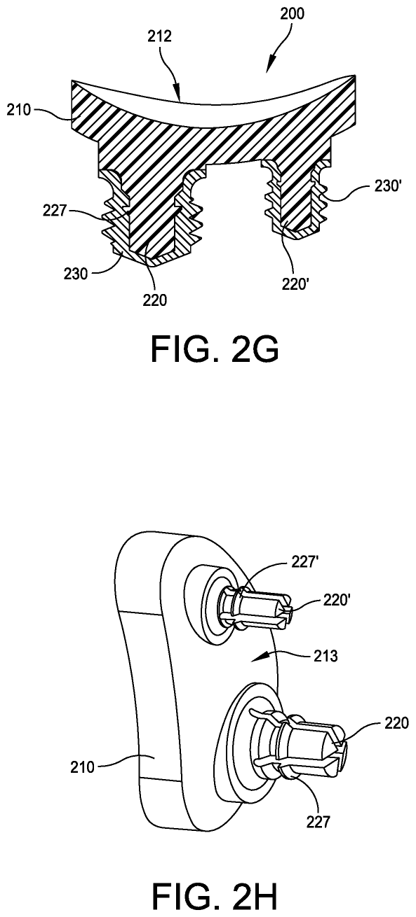

In some embodiments, the substantially radially extending tab 127 can be a single annular continuous collar-like tab on the protrusion 120. In some embodiments, the annular collar-like structure can be discontinuous and be divided into two or more segments, thus, forming two or more radially extending tabs on the protrusion 120. This would be similar to the way the radially extending tabs 227 and 227′ extend around the protrusions 220, 220′ with some discontinuity as shown in FIG. 2H. Either way, as long as the segmented tabs are positioned along an annular collar-like arrangement on the surface of the protrusion 120, the radially extending tabs 127 can engage with one annular groove 137 on the fixation element 130.

In some embodiments, the articular body 110 and the integrally formed radially extending tabs 127 can be formed of, whole or in part, of a high modulus synthetic material. Polymers such as, for example, polyethylene (e.g. ultra-high-molecular-weight polyethylene (UHMWPE)), polyether ether ketone (PEEK), etc. that are selected to provide the desired performance for the articulation surface 112 can be used. All references to UHMWPE herein includes all variants of UHMWPE in orthopedic application such as vitamin E diffused UHMWPE.

The fixation element 130 is formed of a material that is more rigid, i.e., has a higher Young's modulus, than the protrusion 120. According to some embodiments, the fixation element 130 can be metallic and/or ceramic. For example, the fixation element 130 can be made from titanium, stainless steel, an alloy of titanium, and/or an alloy of cobalt-chromium, or a biocompatible ceramic. In some embodiments, the metallic fixation element 130 can be coated with a coating that promotes bone in-growth. An example of such coating material is a porous metallic coating ADAPTIS™ by Wright Medical Technology.

With the fixation element 130 being more rigid than the one or more substantially radially extending tabs 127, when one of the at least one protrusion 120 is inserted into the recess 135 of a fixation element 130, the one or more substantially radially extending tabs 127 elastically deform as the protrusion 120 advances into the recess 135 and snaps back into the original shape when the radially extending tabs 127 reaches the annular groove 137, thus forming the snap-fitting engagement.

As shown in FIGS. 1B and 1C, each of the at least one protrusion 120 defines a longitudinal axis L. To form the snap-fitting engagement, the one or more substantially radially extending tabs 127 extend toward the anchoring surface 113, i.e., in the proximal direction, so that the leading surface 127a of the one or more substantially radially extending tabs 127 form an acute angle a with the longitudinal axis L toward the anchoring surface 113. The leading surface 127a is referred to herein as a “leading” surface because that is the side of the radially extending tabs 127 that is facing the recess 135 of the fixation element 130 when the protrusion 120 and the fixation element 130 engage each other to form the snap-fitting engagement.

The recess 135 comprises a sidewall 136 and the annular groove 137 is cut into the sidewall 136. The annular groove 137 comprises distal face 137a and a proximal face 137b, that correspond to the leading surface 127a, and the trailing surface 127b of the one or more radially extending tabs 127, respectively. The distal face 137a form the acute angle a with the longitudinal axis L toward the anchoring surface 113 when the at least one protrusion 120 is in snap-fitting engagement with the fixation element 130.

The fixation element 130 has an outer diameter of that is between 5-25 mm. Preferably, the outer diameter is between 6.5-20 mm, more preferably between 6.5-15 mm, and most preferably between 9-15 mm. The ranges provided for the outer diameter are inclusive of the endpoints.

In some embodiments, the glenoid implant 100 comprises one protrusion 120 and one fixation element 130.

In some embodiments, each of the at least one protrusion 120 can have a split collet structure to further enhance the snap-fitting engagement between the protrusion 120 and the fixation element 130. The collet structure can be seen in FIG. 2H where examples of such protrusions 220, 220′ on the glenoid implant 200 are illustrated.

In some embodiments, the glenoid implant 100, further comprises one or more additional fixation features 140 extending from the anchoring surface 113 configured to prevent rotation of the articular body 110 with respect to the glenoid when the glenoid implant 100 is implanted in the glenoid. Each of the one or more fixation features 140 can be a structure in the form of a post, as in the illustrated example of FIGS. 1A-1B, a finned anchor, or a keel. The glenoid would be prepared appropriately to receive the one or more fixation features 140 depending on the shape of the fixation features 140.

In the illustrated example, each of the one or more fixation features 140 is a metallic peg 142 that is co-molded with the articular body 110 which is a polymer. As shown in the sectional view in FIG. 1B, the metallic peg 142 comprises a thinner stem portion 142′ onto which the polymer articular body material is co-molded. Preferably, the surface of the stem portion 142′ is textured so that the co-molded interface between the articular body 110 and the stem portion 142′ is stronger. In some embodiments, the surface of the stem portion 142′ can be threaded. The co-molded portion forms a base 122 of the fixation feature 140. In some embodiments, the metallic pegs 142 can be made of such material as Tritanium® by Stryker Corp. which is a porous material, or other porous metallic material, to enhance bone tissue in-growth and, thus, further enhance the fixation of the implant 100 to the glenoid.

In some embodiments of the glenoid implant 100, each of the at least one protrusion 120 has a split collet structure.

The overall outline shape of the glenoid implant body 110 and the curvature of the anchoring surface 113 are configured to match the patient's glenoid. To install the glenoid implant 100, the glenoid surface is prepared with a first hole to receive the fixation element 130 and additional holes or slots positioned around the first hole to match the arrangement of the positions of the one or more fixation features 140 with respect to the fixation element 130 as shown in FIG. 1A.

During the surgical procedure for implanting the glenoid implant 100, after the glenoid is prepared to form a concave surface for receiving the glenoid implant 100, a pilot hole of an appropriate diameter is drilled into the glenoid for receiving the fixation element 130 so that the screw threads 133 can tap into the pilot hole and allow the fixation element 130 to be threaded into the pilot hole. Then, with the distal end 132 of the fixation element 130 facing toward the pilot hole in the glenoid, the fixation element 130 is threaded into the pilot hole until the proximal end 131 of the fixation element is flush with the surface of the glenoid.

When using the embodiment of the fixation element 130 whose longitudinally-extending recess 135 is close-ended, a guide pin can be inserted into bottom of the pilot hole at the center of the pilot hole. Then, the fixation element 130 is placed over the guide pin using the guide hole 138, so that the distal end 132 of the fixation element 130 is facing toward the pilot hole in the glenoid. The fixation element 130 is then slid down the guide pin until the distal end 132 of the fixation element 130 reaches the pilot hole. Then the fixation element 130 is threaded into the pilot hole until the proximal end 131 of the fixation element is flush with the surface of the glenoid.

At some point during these steps, before the implant body 110 can be installed, holes are drilled into the glenoid for receiving the one or more fixation features 140. Next, the implant body 110 is installed by aligning the protrusion 120 with the recess 135 in the fixation element 130 and aligning the one or more fixation features 140 with the corresponding holes prepared in the glenoid then pushing the protrusion 120 into the recess 135 until the snap-fitting engagement is achieved.

One of the benefits of the glenoid implant 100 is that if a revision of the implant is required, the structure of the glenoid implant 100 allows removal of the implant body 110 with minimal damage to the glenoid, if any. To remove the implant body 110, the fixation features 140 can be cut at the co-molded polymer base structure 122. Once the fixation features 140 are cut, the assembly of the implant body 110 and the fixation element 130 can be removed by unscrewing the assembly from the glenoid. Then, the metallic posts 142 can be pulled out of the glenoid.

Referring to FIGS. 2A-2H, according to another embodiment, a glenoid implant 200 is also disclosed. The glenoid implant 200 comprises an articular body 210, two protrusions 220, 220′ and one fixation element 230, 230′ provided for each of the two protrusions. The articular body 210 comprises an articulation surface 212 and an anchoring surface 213 on opposite side of the articular body 210. The wo protrusions 220, 220′ are integrally formed with the articular body and extend from the anchoring surface 213. Each fixation element 230, 230′ have the same structure as the fixation element 130 described above. Each fixation element 230, 230′ comprises a proximal end, a distal end, a longitudinally-extending recess 235, 235′ extending distally from the proximal end, and a screw-threaded external surface.

As in the glenoid implant embodiment 100, the two protrusions 220, 220′ and the corresponding fixation elements 230, 230′ are configured for snap-fitting engagement when the two protrusions are inserted into the recess 235, 235′ of the corresponding fixation element 230, 230′.

In some embodiments, each of the protrusions 220, 220′ can have a split collet structure to further enhance the snap-fitting engagement between the protrusions 220, 220′ and the fixation elements 230, 230′. The collet structure can be seen in FIG. 2H where examples of such protrusions 220, 220′ on the glenoid implant 200 are illustrated.

FIGS. 2A and 2B are labeled to show the superior side and the inferior side of the implant 200 indicating the implanted orientation in the patient. To accommodate the physical dimensions of a glenoid where the inferior portion is larger than the superior portion, the superiorly located fixation element 230′ of the glenoid implant 200 has a smaller diameter than the inferiorly located fixation element 230.

Referring to FIG. 2E, similar to the protrusions 120 in the glenoid implant 100, each of the two protrusions 220, 220′ in the glenoid implant 200 comprises one or more substantially radially extending tabs 227, 227′, respectively, provided on exterior surfaces of the protrusions. Each fixation element 230, 230′ are provided with a longitudinally-extending recess 235, 235′. The longitudinally-extending recess 235, 235′ can be open-ended and in some embodiments, it can be close-ended.

Each of the recesses 235, 235′ in each fixation elements 230, 230′ comprises an annular groove 237, 237′, where the one or more substantially radially extending tabs 227, 227′ and the annular grooves 237, 237′ form the snap-fitting engagement when the protrusions 220, 220′ are pushed into their respective recesses 235, 235′. As described above in conjunction with the glenoid implant 100, the snap-fitting engagement can be formed using a c-clip or an O-ring captured between the protrusions 220, 220′ and their respective recesses 235, 235′.

Each of the two protrusions 220, 220′ defines a longitudinal axis L and the corresponding one or more substantially radially extending tabs 227, 227′ extend toward the anchoring surface 213 so that a leading surface 227a, 227a′ of the one or more substantially radially extending tabs form an acute angle a with the longitudinal axis L toward the anchoring surface 213.

The recess 235, 235′ in each fixation element 230, 230′ comprises a sidewall 236, 236′ and the annular groove 237, 237′ is cut into the sidewall, wherein the annular groove comprises a distal face 237a, 237a′ and a proximal face 237b, 23b′ that correspond to the leading surface 227a, 227a′, and the trailing surface 227b, 227b′ of the radially extending tabs 227, respectively. The leading surface 227a, 227a′ and the distal face 237a, 237a′ form an angle with the longitudinal axis L toward the anchoring surface 213 that is substantially the acute angle a when the two protrusions 220, 220′ are in snap-fitting engagement with the corresponding fixation elements 230, 230′. Each of the fixation elements 230, 230′ further comprise a guide hole 238, 238′, respectively, that is used to guide the fixation element over a guide pin during the implant surgical procedure which is described below.

Because of the geometry of the glenoid is not circular but asymmetric, one of the two fixation elements 230, 230′ is the larger diameter fixation element that satisfy the outer diameter requirement of between 5-25 mm. Preferably, the outer diameter is between 6.5-20 mm, more preferably between 6.5-15 mm, and most preferably between 9-15 mm. The outer diameter ranges provided are inclusive of the endpoints. In the illustrated example glenoid implant 200, the fixation element 230 is the larger diameter fixation element.

In some embodiments, the articular body 210 and the integrally formed radially extending tabs 227, 227′ can be formed of, whole or in part, of a high modulus synthetic material. Polymers such as, for example, polyethylene (e.g. ultra-high-molecular-weight polyethylene (UHMWPE)), polyether ether ketone (PEEK), etc. that are selected to provide the desired performance for the articulation surface 212 can be used. All references to UHMWPE herein includes all variants of UHMWPE in orthopedic application such as vitamin E diffused UHMWPE.

The fixation elements 230, 230′ are formed of a material that is more rigid, i.e., has a higher Young's modulus, than the protrusions 220, 220′. According to some embodiments, the fixation element 230, 230′ can be metallic and/or ceramic. For example, the fixation element 230 can be made from titanium, stainless steel, an alloy of titanium, and/or an alloy of cobalt-chromium, or a biocompatible ceramic. In some embodiments, the metallic fixation element 230 can be coated with a coating that promotes bone in-growth. An example of such coating material is a porous metallic coating ADAPTIS TM by Wright Medical Technology.

In some embodiments, the glenoid implant 200 further comprises one or more fixation features 240 (not shown), like the fixation features 140 described above in connection with the glenoid implant 100, including all of the variations and options.

During the surgical procedure for implanting the glenoid implant 200, after the glenoid is prepared to form a concave surface for receiving the glenoid implant 200, a pilot hole of an appropriate diameter is drilled into the glenoid for each of the fixation elements 230, 230′ so that the screw threads 233, 233′ can tap into the pilot hole and allow the fixation elements 230, 230′ to be threaded into the corresponding pilot hole. Then, with the distal ends 232, 232′ of the fixation elements 230, 230′ facing toward the corresponding pilot hole in the glenoid, the fixation elements 230, 230′ are threaded into the pilot holes until the proximal ends 231, 231′ of the fixation elements are flush with the surface of the glenoid.

When using the embodiment of the fixation elements 230, 230′ whose longitudinally-extending recesses 235, 235′ are close-ended, a guide pin can be inserted into the bottom of each of the pilot holes at the center of the pilot holes. Then, the fixation elements 230, 230′ are placed over the guide pins using the guide holes 238, 238′ one at a time so that the distal ends 232, 232′ of the fixation elements 230, 230′ are facing toward the pilot holes in the glenoid. Each of the fixation elements 230, 230′ are then slid down their corresponding guide pin until the distal ends 232, 232′ of the fixation elements 230, 230′ reach their respective pilot hole. Then the fixation elements 230, 230′ are threaded into the pilot holes until the proximal ends 231, 231′ of the fixation elements are flush with the surface of the glenoid.

At some point during these steps, before the implant body 210 can be installed, holes are drilled into the glenoid for receiving the one or more fixation features 240. Next, the implant body 210 is installed by aligning the protrusions 220, 220′ with the recesses 235, 235′ in the fixation elements 230, 230′ and aligning the one or more fixation features 240 with the corresponding holes or slots prepared in the glenoid then pushing the protrusions 220, 220′ into the recesses 235, 235′ until the snap-fitting engagement is achieved.

In some embodiments, the glenoid implant 200 can be offered in a number of different sizes for the implant body 210 but share the same spacing between the two protrusions 220, 220′. Then, glenoid implants 200 having different size implant body 210 can be tried on a given glenoid by preparing one pair of holes for receiving the corresponding fixation elements 230, 230′.

In some situations where an RSA (reverse shoulder arthroplasty) baseplate is to be placed over the glenoid implant 200, depending on the particular structure and configuration of the RSA baseplate, the glenoid implant 200 can be configured to have both fixation elements 230, 230′ or just one as appropriate. For example, an embodiment for use with the RSA baseplate in Stryker's ReUnion RSA system where only the inferior half of the glenoid is reamed to receive the RSA baseplate, the glenoid implant 200 can be configured to have only the superiorly positioned fixation element 230′ because the bottom inferior half of the glenoid implant 200 would be fixed by the bone screws that secure the RSA baseplate by traversing through the implant body 210 and into the underlying glenoid bone.

In some embodiments, the surgeon can trial off of the fixation elements 230, 230′ that are placed in the glenoid and offer a biased insert to tweak anteversion.

The glenoid implants of the present disclosure are designed to provide many improved characteristics over the conventional baseplate designs. Some of the benefits include, but not limited to: superior fixation strength; low cost; revisable with little to no bone loss; no baseplate, which lessens the stiffness of the implant and potentially provide clinical benefit; revisable from TSA (total shoulder arthroplasty) to RSA. The glenoid implants of the present disclosure also should exhibit reduced stiffness that is below any metal backed glenoid implants, which is expected to provide improved primary fixation to the glenoid and extend the life of the implant.

Although the devices, kits, systems, and methods have been described in terms of exemplary embodiments, they are not limited thereto. Rather, the appended claims should be construed broadly, to include other variants and embodiments of the devices, kits, systems, and methods, which may be made by those skilled in the art without departing from the scope and range of equivalents of the devices, kits, systems, and methods.

1-22. (canceled)

23. A glenoid implant comprising:
an articular body comprising an articulation surface and an anchoring surface on opposite side of the articular body;
two protrusions that are integrally formed with the articular body and extending from the anchoring surface; and
one fixation element provided for each of the two protrusions, wherein each fixation element comprising:
a proximal end, a distal end, a longitudinally-extending recess extending distally from the proximal end, and a screw-threaded external surface,
wherein the two protrusions and the corresponding fixation element are configured for snap-fitting engagement when the two protrusions are inserted into the close-ended recess of the corresponding fixation element.

24. The glenoid implant of claim 23, wherein the longitudinally-extending recess is a close-ended recess.

25. The glenoid implant of claim 23, wherein each of the two protrusions comprises one or more substantially radially extending tabs provided on exterior surface of the protrusion and the longitudinally-extending recess in each fixation element comprises an annular groove, wherein the one or more substantially radially extending tabs and the annular groove form the snap-fitting engagement.

26. The glenoid implant of claim 23, wherein each of the two protrusions defines a longitudinal axis and the corresponding one or more substantially radially extending tabs extend toward the anchoring surface so that a leading surface of the one or more substantially radially extending tabs form an acute angle with the longitudinal axis toward the anchoring surface.

27. The glenoid implant of claim 23, wherein the longitudinally-extending recess in each fixation element comprises a sidewall and the annular groove is cut into the sidewall, wherein the annular groove comprises a distal face that corresponds to the leading surface, and the distal face form an angle with the longitudinal axis toward the anchoring surface that is substantially the acute angle when the two protrusion are in snap-fitting engagement with the corresponding fixation elements.

28. The glenoid implant of claim 23, wherein the fixation elements and the articular body comprises different materials.

29. The glenoid implant of claim 23, wherein the fixation elements are formed of a material having higher Young's modulus than that of the articular body.

30. The glenoid implant of claim 23, wherein the articular body is made of a high modulus polymer.

31. The glenoid implant of claim 30, wherein the polymer is UHMW polyethylene.

32. The glenoid implant of claim 29, wherein the fixation element is formed of a metal alloy or biocompatible ceramic.

33. The glenoid implant of claim 23, further comprising one or more fixation features extending from the anchoring surface configured to engage the glenoid when the glenoid implant is implanted in the glenoid.

34. The glenoid implant of claim 33, wherein the one or more fixation features extending from the anchoring surface can comprise a post, a finned anchor, or a keel.

35. The glenoid implant of claim 33, wherein each of the one or more fixation features is a metallic post that is co-molded with the articular body.

36. The glenoid implant of claim 35, wherein the post further comprises one or more bone.

37. The glenoid implant of claim 23, wherein each of the two protrusions has a split collet structure.

38. The glenoid implant of claim 23, wherein at least one of the fixation elements has an outer diameter that is between 5-25 mm, inclusive.

39. The glenoid implant of claim 23, wherein at least one of the fixation elements has an outer diameter that is between 6.5-20 mm, inclusive.

40. The glenoid implant of claim 23, wherein at least one of the fixation elements has an outer diameter that is between 6.5-15 mm, inclusive.

41. The glenoid implant of claim 23, wherein at least one of the fixation elements has an outer diameter that is between 9-15 mm, inclusive.
